(19) United States
(12) Patent Application Publication
(10) Pub. No.: US 2026/0157860 A1
(43) Pub. Date: Jun. 11, 2026

(54) ONE-STEP INSTRUMENT FOR IMPLANTING STEMLESS PROSTHESES

(71) Applicant: Zimmer, Inc, Warsaw, IN (US)

(72) Inventor: Cody Johnathon Holmes, Warsaw, IN (US)

(21) Appl. No.: 19/181,062

(22) Filed: Apr. 16, 2025

Related U.S. Application Data
(60) Provisional application No. 63/651,568, filed on May 24, 2024.

(51) Int. Cl. A61F 2/46; A61F 2/40

(52) U.S. Cl. CPC A61F 2/46; A61F 2/40

(57) ABSTRACT

An instrument for preparing a bone to receive a prosthesis comprises a shaft extending along a central axis from a distal end to a proximal end, a distal reaming element comprising a boss reamer located at the distal end, and a broaching element located proximally of the distal reaming element and configured to rotate about the shaft, wherein the broaching element comprises a plurality of spokes extending radially outward of the distal reaming element. A method of modifying a humeral head to receive a stemless humeral implant using an instrument having an integrated broach and reamer comprises resecting a bone to form a resected surface, inserting the instrument into the resected surface, rotating a shaft of the instrument to rotate a distal reamer and form a center boss, and advancing a broach of the instrument into the resected surface to form a plurality of slots extending from the center boss.

### CLAIM OF PRIORITY

This application claims the benefit of U.S. Provisional Patent Application Ser. No. 63/651,568, filed on May 24, 2024, the benefit of priority of which is claimed hereby, and which is incorporated by reference herein in its entirety.

### TECHNICAL FIELD

The present disclosure is generally directed to, but not by way of limitation, systems, devices and methods for performing medical procedures, such as partial and total shoulder arthroplasties. More specifically, but not by way of limitation, the present disclosure is directed to medical instruments used to perform reverse shoulder arthroplasties, such as broaches and reamers used to shape bone to receive a bone anchor.

### BACKGROUND

The shoulder joint includes a humerus bone and a scapula bone, which cooperate to provide range of motion of the humerus relative to the scapula during movement of a human arm. Specifically, a proximal end of the humerus including a humeral head is disposed adjacent to a glenoid fossa of the scapula and is permitted to move relative to the glenoid fossa to provide a range of motion to the humerus relative to the scapula.

Joint replacement surgery, such as a partial or total shoulder arthroplasty, may be required or desired when the shoulder joint causes pain during use or is otherwise damaged. For example, the shoulder joint may be damaged due to osteoarthritis, whereby progressive wearing away of cartilage results in bare bone being exposed within the shoulder joint. Under such circumstances, it is often necessary or desirable to undergo a partial or total shoulder arthroplasty in order to relieve pain and increase the range of motion of the humerus by rebuilding portions of the shoulder joint.

In performing a total shoulder arthroplasty, a surgeon resects a portion of the proximal end of the humerus that is received by the glenoid fossa, e.g., the humeral head. Once the proximal end of the humerus is resected, the surgeon can then ream the humerus to access the humeral canal. Providing access to the humeral canal allows the surgeon to insert an anchor component, such as a stemmed prosthesis, into the humeral canal. In anatomic arthroplasty procedures, a prosthetic humeral head is attached to an elongate stemmed anchor component.

For example, a hemispheric-shaped prosthetic humeral head can then be attached to a proximal end of the anchor component such that the resected portion of the humerus is replaced by the prosthetic humeral head. If total shoulder arthroplasty is to be performed, the surgeon can likewise replace a portion of the glenoid fossa with a prosthetic bearing component to provide a bearing surface against which the prosthetic humeral head can be configured to articulate. In a reverse shoulder arthroplasty, the prosthetic humeral head component is attached to the scapula and the prosthetic bearing component is attached to the humerus. In a partial shoulder arthroplasty, only one of the humeral head and scapula is repaired with a prosthetic implant and the natural bone structure is used on the other bone. Upon completion of the shoulder arthroplasty, pain is typically alleviated, and the patient is provided with an increased range of motion at the shoulder joint.

While conventional shoulder prosthetics used during shoulder arthroplasty adequately provide the patient with an increased range of motion, conventional shoulder prosthetics typically involve insertion of a stem, e.g., a stemmed prosthesis or stemmed anchor, into the humeral canal of the humerus, thereby increasing the overall weight, size, and cost of the humeral component. Furthermore, because the surgeon inserts the stem of the stemmed prosthesis into the humeral canal, the surgical procedure is somewhat complex, as the surgeon first resects the humeral head of the humerus, and subsequently performs one or both of a broaching operation and a reaming operation on the humeral canal prior to inserting the stem of the stemmed prosthesis into the humeral canal. As such, care must be exorcised to not unduly harm the integrity of the humerus and produce additional weaknesses from the removal of bone. Increasing the complexity of the joint-replacement surgery also increases the time in which the surgeon spends in performing the procedure and therefore increases the overall cost of the procedure. Finally, insertion of the stem into the humerus can result in additional bone removal, thereby increasing trauma and post-operative pain.

Examples of humeral broaches are described in Pub. No. US 2020/0315807 to Hatzidakis, titled “Shoulder “Arthroplasty Implant System”; Pub. No. WO/2007/109340 to Reubelt, titled “Femoral and Humeral Stem Geometry and Implantation Method for Orthopedic Joint Reconstruction”; and Pub. No. US 2021/0030552 to Terrill, titled “Keeled Glenoid Implant.”

### OVERVIEW

The present inventors have recognized, among other things, that problems to be solved in preparing a shoulder joint to receive a prosthetic component is the desirability of preserving bone matter, particularly in the humerus. As mentioned above, implantation of an elongate stem into the humeral canal can be a complex procedure benefiting from care and caution during the implantation procedure. In order to limit potentially adverse effects of inserting an elongate stem into the humeral canal, stemless humeral implants have been developed where the use of elongate stems are avoided by using anchoring components that attach to bone matter near the humeral head at the anatomic neck. In some stemless configurations, a tray can be attached to a stemless anchoring component and the prosthetic component can be attached to the tray.

Stemless anchoring implants using trays are particularly beneficial in reverse shoulder systems where a prosthetic glenoid component is attached to the humeral bone. In such cases, it can be desirable to vary the depth of the implantation of the tray to control joint tightness, whereas in anatomic shoulder systems the depth of implantation of the prosthetic humeral head is typically not varied since, for example, the prosthetic humeral head is sized to mate with the resected bone surface, such as to engage with cortical bone. Furthermore, in anatomic shoulder arthroplasty, the depth of resection is typically less than the depth of resection for a reverse shoulder arthroplasty, leaving more bone to support the prosthetic humeral head compared to the use of a tray to support the prosthetic bearing in reverse shoulder arthroplasties. Thus, for stemless reverse shoulder implants, a tray that receives the prosthetic component, e.g., the prosthetic bearing, can be implanted flush against the resected bone surface in an onlay configuration or can be recessed into the resected bone surface in an inlay configuration. As such, the bone is modified differently to prepare the inlay anchor component than the onlay anchor component. For onlay configurations, a reamer procedure is typically followed by a broaching procedure to prepare the bone for the stemless anchoring component. For inlany configurations, the reaming and broaching procedures are typically followed by a second reaming procedure to prepare the bone for the tray.

Several factors can affect whether an inlay anchor component or an onlay anchor component is used, such as how much bone is to be removed with the resection to remove diseased or damaged bone, and other factors, such as surgeon preference. Furthermore, the laxity of the shoulder joint, e.g., the tension produced in the joint by ligaments and other soft tissue, is additionally taken into account. Sometimes, the decision to use an onlay or inlay tray configuration is made intra-operatively, making the need for being readily able to perform bone modifications in either case desirable. As such, the present inventor has recognized that preparation of bone to receive stemless implants to accommodate both onlay and inlay implants can be complicated. There is a need, therefore, for instruments for implanting stemless humeral implants that are simple and easy to assemble and operate,

The present subject matter can provide solutions to these and other problems, such as by providing a bone modification instrument that can integrate reaming and broaching operations into a single device that can be operated in a single step. In examples, a distal reamer can be configured to engage bone, and a broach instrument can engage with bone proximal of the distal reamer. The broach instrument can rotate relative to the distal reamer such that as the distal reamer advances into bone, the broach can be pushed into bone without rotation. Furthermore, in additional examples, a proximal reamer can be located on the shaft of the distal reamer proximal of the broach instrument. As such, the instruments of the present disclosure can be configured to produce multiple, different types of bone modifications using a single instrument in a single procedure at different axial positions and with different radial reach. The subject matter of the present application can be applied to both onlay and inlay bone preparation. The subject matter of the present application can be applied to both stemmed and stemless humeral prosthetic components, as well as other components that can have different configurations of differing thicknesses.

In an example, an instrument for preparing a bone to receive a prosthetic implant can comprise a shaft extending along a central axis from a distal end to a proximal end, a distal reaming element located at the distal end of the shaft, wherein the distal reaming element comprises a boss reamer, and a broaching element located proximally of the distal reaming element, the broaching element configured to rotate about the shaft, wherein the broaching element comprises a plurality of spokes extending radially outward of the distal reaming element.

In another example, a method of modifying a humeral head to receive a stemless humeral implant using an instrument having an integrated broach and reamer can comprise resecting a bone to form a resected surface, inserting the instrument into the resected surface, rotating a shaft of the instrument to rotate a distal reamer and form a center boss, and advancing a broach of the instrument into the resected surface to form a plurality of slots extending from the center boss.

### BRIEF DESCRIPTION OF THE DRAWINGS

FIG. 1 is a side view of an arthroplasty system for an anatomic shoulder implant.

FIG. 2 is a side view of an arthroplasty system for a reverse shoulder implant in an onlay configuration.

FIG. 3 is a side view of an arthroplasty system for a reverse shoulder implant in an inlay configuration.

FIG. 4 is a top perspective view of a stemless anchor component that can be used with the arthroplasty systems of FIG. 1, FIG. 2 and FIG. 3.

FIG. 5 is a bottom perspective view of the stemless anchor component of FIG. 4.

FIG. 6 is a top view of the stemless anchor component of FIG. 4 and FIG. 5.

FIG. 7 is a side view of a prosthesis implant instrument of the present disclosure comprising a distal reamer, a middle broach, a proximal reamer and a coupler.

FIG. 8 is an exploded side view of a prosthesis implant instrument of FIG. 7.

FIG. 9 is a perspective view of the prosthesis implant instrument of FIG. 7.

FIG. 10 is a perspective view of the distal reamer of FIG. 7 comprising a shaft and a distal reaming head.

FIG. 11 is a cross-sectional view of the distal reamer of FIG. 10.

FIG. 12 is a perspective view of the middle broach of FIG. 7 comprising a hub and radial cutting blades.

FIG. 13 is a top view of the middle broach of FIG. 12.

FIG. 14 is a cross-sectional view of the middle broach of FIG. 12.

FIG. 15 perspective view of the proximal reamer of FIG. 7 comprising a hub and a proximal reaming head.

FIG. 16 is a top view of the proximal reamer of FIG. 15.

FIG. 17 is a cross-sectional view of the proximal reamer of FIG. 15.

FIG. 18 perspective view of the coupler of FIG. 7 comprising a proximal coupler portion and a distal coupler portion.

FIG. 19 is a side view of the coupler of FIG. 18.

FIG. 20 is a cross-sectional view of the coupler reamer of FIG. 18.

FIG. 21 is a cross-sectional view of the prosthesis implant instrument of the present disclosure in an exploded state to show a coupler, a proximal reamer, a middle broach and a distal reamer.

FIG. 22 is a cross-sectional view of the prosthesis implant instrument of the present disclosure in an assembled state.

FIG. 23 is a line diagram illustrating operations of methods of the present disclosure relating to implanting stemless prostheses with a one-step bone modification instrument.

### DETAILED DESCRIPTION

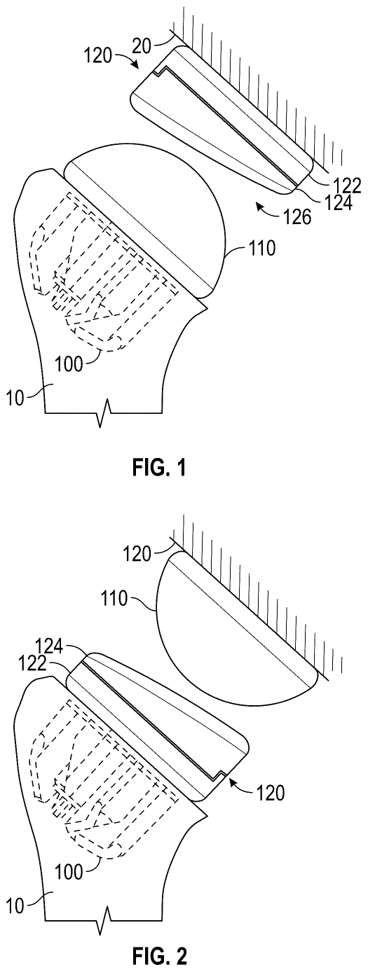

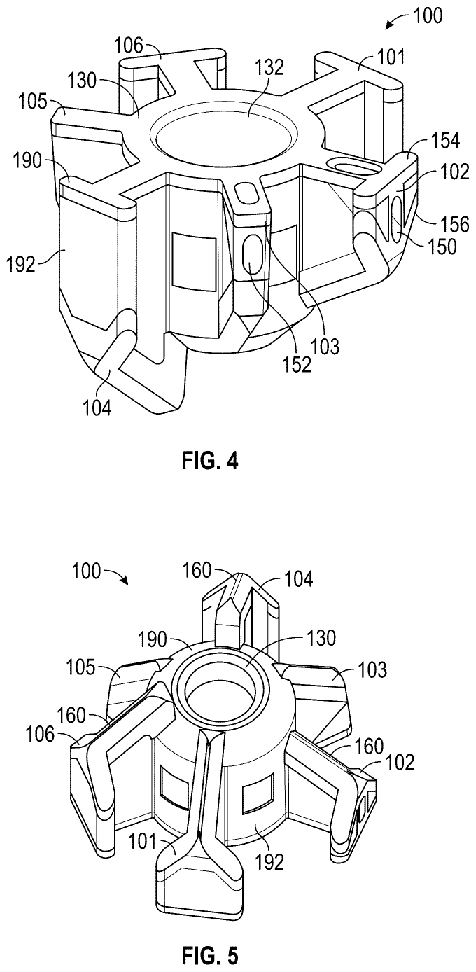

FIG. 1 shows an arthroplasty system for an anatomic shoulder implant that can be used with the instruments of the present disclosure. The arthroplasty system can include humeral anchor 100 configured to be mounted to humerus 10. Prosthetic component 110, which can be referred to as a humeral head when attached to humerus 10, can be mounted to humeral anchor 100. Prosthetic component 110 can include a post or cone, which can be a male taper, extending from a bottom surface which can mount to, e.g., be inserted into a socket, e.g., cylindrical bore 132 of FIG. 4, of humeral anchor 100.

The arthroplasty system can further include a complimentary component, such as articulating component 120 including tray 122 and bearing 124. Tray 122 can be concave and recessed to form a compartment for receiving bearing 124. Bearing 124 can include concave bearing surface 126 which can receive prosthetic component 110 when implanted. Articulating component 120 can be configured to be mounted to scapula 20 proximate to a glenoid cavity.

Prosthetic component 110 can be configured to articulate against concave bearing surface 126 when implanted. FIG. 1 shows an example of a two-piece articulating component that can be converted for use in anatomic and reverse shoulder implants. However, the present disclosure can be used with other types of scapula or glenoid components. In examples, some glenoid components can comprise a bone-facing surface that can mate with a prepared bone surface of the scapula. The bone-facing surface can have one or more pegs or posts that can be inserted into bores formed in the prepared bone surface. The glenoid component can include a bearing surface or articulation surface opposite the bone-facing surface to engage with an anatomic humeral head or a prosthetic humeral head. The bearing surface can have a radius of curvature allowing for joint movement like that of a native gleno-humeral joint. In examples, glenoid components can be fabricated from polymer material, such as by being a monolithic, uni-body polymer component. In examples, articulating components of the present disclosure can comprise or be similar to those described in U.S. Pat. No. 8,425,614 B2 to Winslow et al., titled “Modular Center Peg Glenoid,” the contents of which are hereby incorporated by this reference.

FIG. 2 shows an arthroplasty system for a reverse shoulder implant in an onlay configuration. Here, prosthetic component 110 can be mounted to scapula 20, while articulating component 120 can be mounted to humerus 10. When attached to the glenoid of scapula 10, prosthetic component 110 can be referred to as a glenosphere. A post extending from a bottom of tray 122 can be mounted to a socket, e.g., cylindrical bore 132 of FIG. 4, of humeral anchor 100. Thus, humeral anchor 100 can be configured to receive both prosthetic component 110 and articulating component 120 in anatomic and reverse shoulder arthroplasties, respectively.

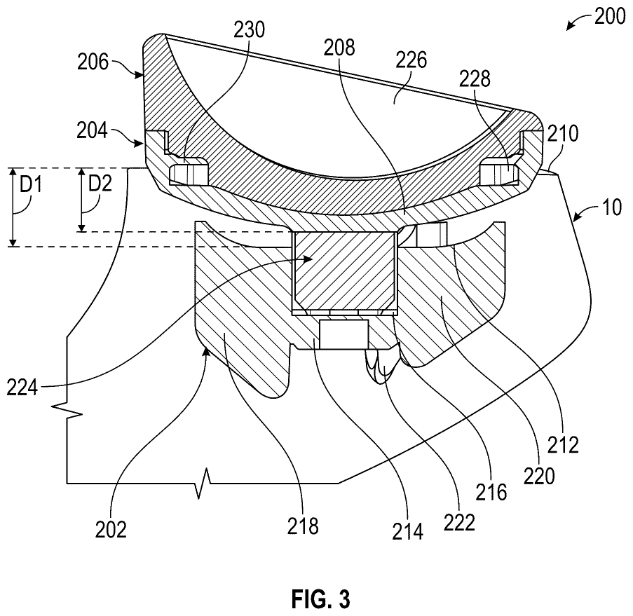

FIG. 3 shows arthroplasty system 200 for a reverse shoulder implant in an inlay configuration. Arthroplasty system 200 can comprise stemless anchor 202, tray 204 and articulating component 206. As with FIG. 2, prosthetic component 110 can be mounted to scapula 20 (FIG. 2), while articulating component 206 can be mounted to humerus 10. However, rather than tray 204 being mounted to humerus 10 so that bottom surface 208 of tray 204 is positioned on or in close proximity to resected surface 210, tray 204 can be recessed into resected surface 210. Bottom surface 208 can be curved or concave to sit into a pocket made in resected surface 210. Thus, top surface 212 of stemless anchor 202 can be configured to sit below resected surface 210 distance D1 and bottom surface 208 of tray 204 can be configured to sit distance D2 below resected surface 210. D1 can be greater than D2.

Stemless anchor 202 can comprise hub 214 having socket 216, fin 218, fin 220 and fin 222. Tray 204 can comprise bottom surface 208 from which peg 224 extends. Articulating component 206 can include bearing surface 226 and attachment features 228. Fin 218, fin 220 and fin 222 can be configured to engage with periphery cancellous bone near the cortical wall, but would typically fall short of interacting with or contacting cortical bone. Peg 224 can be positioned within socket 216 to attach tray 204 to stemless anchor 202. Attachment features 228 of articulating component 206 can engage with attachment features 230 on tray 204 to attach articulating component 206 to tray 204.

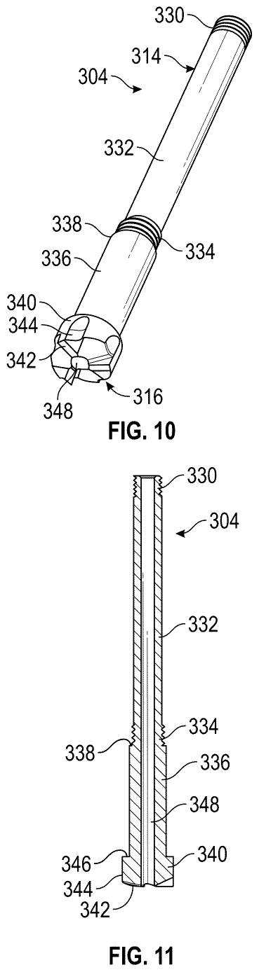

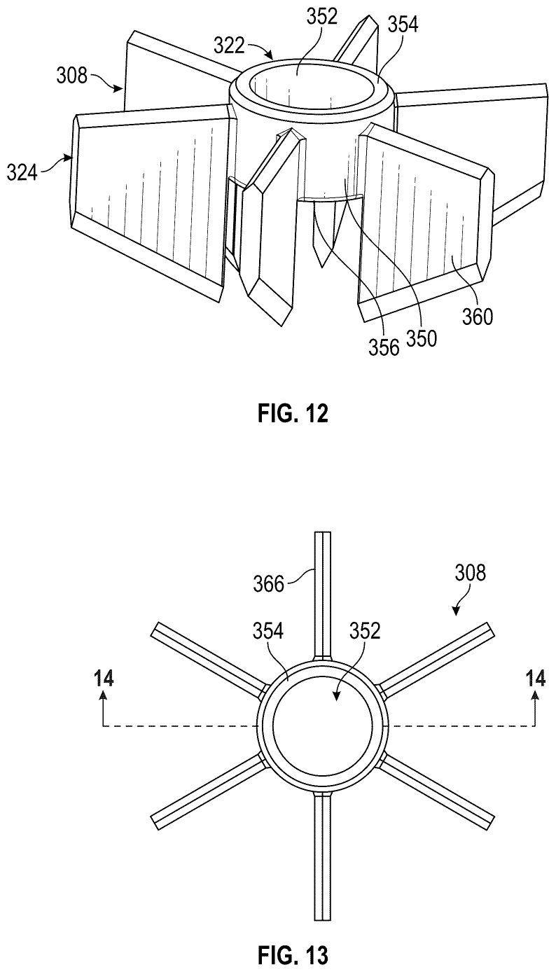

In examples, bottom surface 208 can have the same or similar shape as the shape of blades 321 of proximal reamer 306 (FIG. 15 through FIG. 17). In particular, the concave profile reamed by proximal reamer 306 can match the convex shape of bottom surface 208. In examples, fins of hub 214 can have the same or similar shape as blades 324 of middle broach 308 (FIG. 12 through FIG. 14). In examples, hub 214 can have the same or similar shape as distal reaming head 316 (FIG. 10 and FIG. 11). As such, with the present disclosure, prosthesis implant instrument 300 can perform bone modifications at different axial levels oblique to resected surface 210 and with differing radial extent in a single operation or step to modify humerus 10 to receive stemless anchor 202 and tray 204.

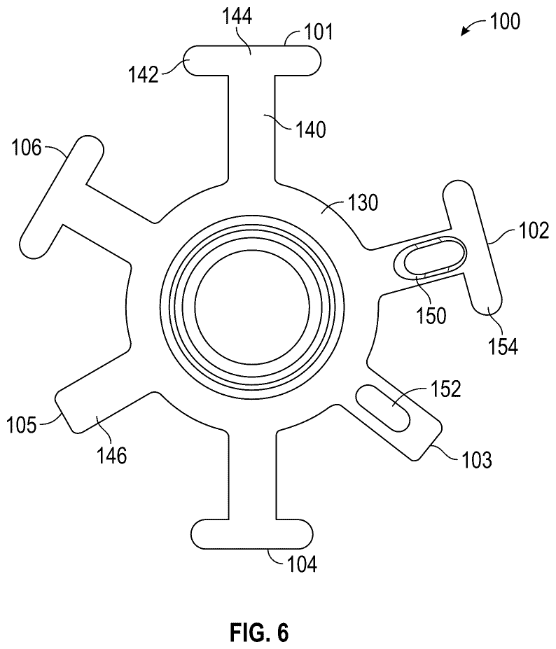

FIG. 4, FIG. 5, and FIG. 6 show details of humeral anchor 100 that can be implanted using the one-step instruments of the present disclosure. FIG. 4 is a top perspective view of humeral anchor 100. FIG. 5 is a bottom perspective view of humeral anchor 100. FIG. 6 is a top view of humeral anchor 100. Humeral anchor 100 of FIG. 4-FIG. 6 is for a left arm. For a right arm, the design of humeral anchor 100 would be a mirror image of the design shown.

Humeral anchor 100 can comprise an embodiment of stemless anchor 202 of FIG. 3.

Humeral anchor 100 can include central hub 130 having fin 101 through fin 106 extending radially from the outer surface walls of central hub 130. Central hub 130 can include a cylindrical bore 132, which can include a female taper, configured to receive the post or cone of either of prosthetic component 110 or articulating component 120. (FIG. 1 and FIG. 2). In examples, the top surface of humeral anchor 100 defines a planar surface where central hub 130 and fin 101 through fin 106 have co-planar upper surfaces. In other examples, the upper surfaces can be parallel, yet offset. As shown in FIG. 3, the upper surface of central hub 130 can be recessed below the upper surfaces of fin 101 through fin 106 to allow for inlay configurations.

Each of fin 101 through fin 106 can have a different height from their top surfaces to their respective bottom edges. Also, each of fin 101 through fin 106 can have a varying radial length from the outer surface of central hub 130 to their outermost surface. Moreover, fin 101 through fin 106 can be arranged in an asymmetric pattern as viewed from the top of humeral anchor 100, as shown in FIG. 6. The sizes and positions of fin 101 through fin 106 are designed to take advantage of the anatomy of the humerus without impinging on the cortical bone. For example, fin 101 through fin 106 can be sized and positioned so that the fins are as long as possible without touching or impinging the cortical bone. Thus, a stemless anchor can be configured to increase surface area engagement with cancellous bone in the metaphysis region of the humeral bone without extending into the diaphysis regions of the humeral bone.

In examples, one or more of fin 101 through fin 106 can include a T-shaped cross section. For example, in this embodiment, fin 101, fin 102, fin 104, and fin 106 include a T-shape. The T-shaped fins can include first portion 140 extending from central hub 130 and second portion 142 perpendicular to first portion 140. Outer flat surface 144 of second portion 142 can provide additional stability within the bone. In other examples not illustrated, fin 101 through fin 106 can be configured to not have any T-shaped tips.

Further, one or more of fin 101 through fin 106 can include a straight shape without the T-portion. For example, fin 103 and fin 105 can include a straight shape including single portion 146 extending from central hub 130. The fins without the T-shape can be located so as not to impinge on the cortical bone of the humerus.

In one embodiment, one or more of fin 101 through fin 106 can include suture hole 150 and suture hole 152 configured to receive a suture. For example, suture hole 150 and suture hole 152 can include a bore extending from top surface 154 of the fin to outer side surface 156 of the fin. In examples, fin 102 and fin 103 can include suture hole 150 and suture hole 152, respectively. Fin 102 and fin 103 are on the side of humeral anchor 100 that will be mounted on the anterior side of the humerus so as to attach the suture to soft tissue.

Referring to FIG. 3 and FIG. 4, humeral anchor 100 can be formed of a titanium metal frame and can include porous metal portions or coatings for biologic bony ingrowth. For example, humeral anchor 100 can be manufactured utilizing additive manufacturing to incorporate a porous metal. Titanium frame 190 can be provided and porous metal 192 can be added at certain portions, for example, at the outer surface of central hub 130 and at the side walls and end surfaces of fin 101 through fin 106. In this example, no porous metal is located on bottom edges 160 of the fins or the bottom edge of central hub 130 to allow for an easier revision process if needed.

In the illustrated example, one or more of fin 101 through fin 106 can include bottom edge 160. The distal tip of each of bottom edge 160 can be V-shaped. Bottom edge 160 at the bottom of fin 101 through fin 106 allow can be sharpened to allow for self-punching during implantation. Also, bottom edge 160 of each of fin 101 through fin 106 has a sloped shape where the section of bottom edge 160 closest to central hub 130 is lower than the outer tip of the fin so that the bottom edge slopes upward from central hub 130 to the outer tip of the fin. Also, as noted above, each of fin 101 through fin 106 can have a different radial length extending from central hub 130 and can have different heights extending from a top surface of the fin to bottom edge 160 of the fin. The devices and instruments of the present disclosure can facilitate implantation of humeral anchor 100 by providing an instrument having a combined reamer for forming a distal boss to receive central hub 130 and a broach to produce radial slots for receiving fin 101 through fin 106 in a single step.

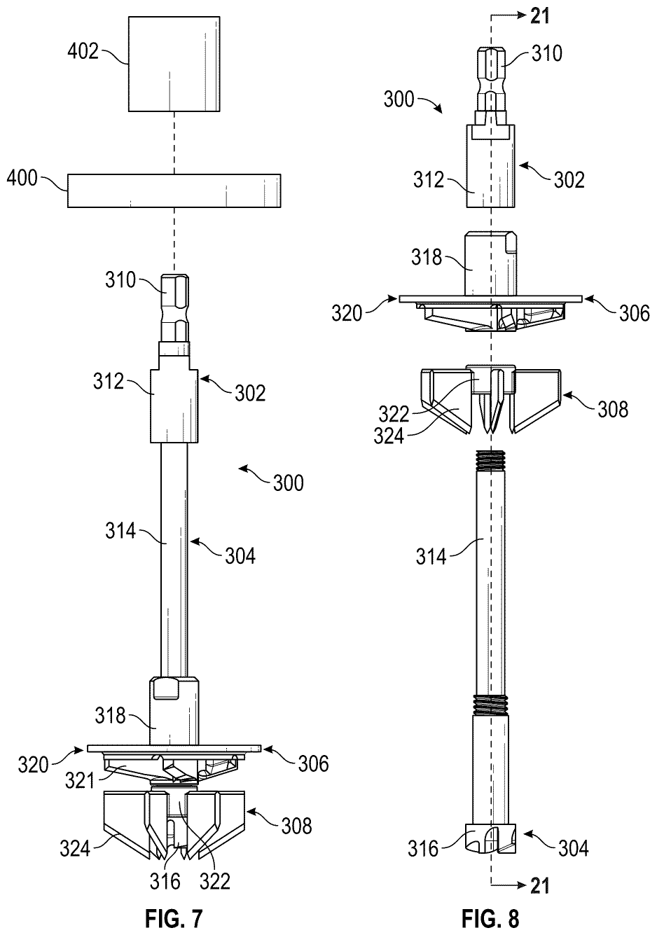

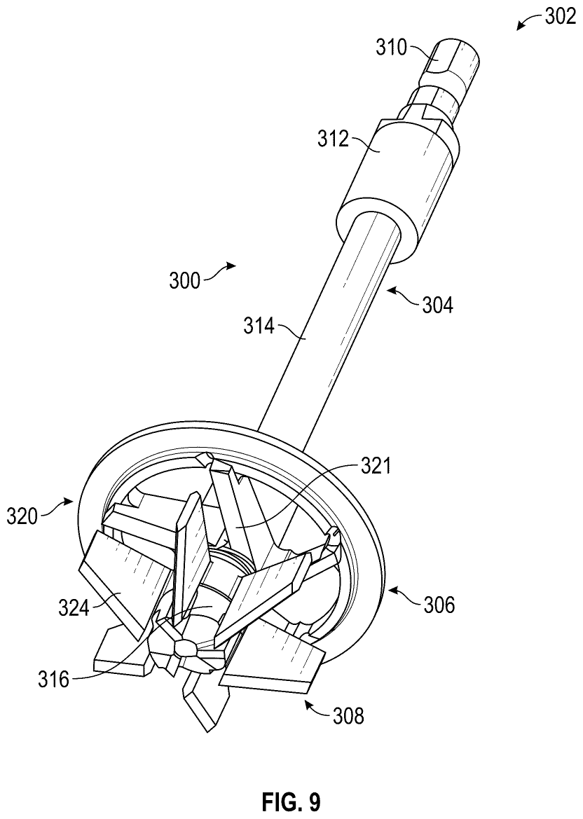

FIG. 7 is a side view of prosthesis implant instrument 300 of the present disclosure comprising coupler 302, distal reamer 304, proximal reamer 306 and middle broach 308. FIG. 8 is an exploded side view of prosthesis implant instrument 300 of the present disclosure. FIG. 9 is a perspective view of prosthesis implant instrument 300 of FIG. 7. As shown schematically in FIG. 7, prosthesis implant instrument 300 can be connected to strike plate 400 and drive tool 402. FIG. 7 through FIG. 9 are discussed concurrently.

Coupler 302 can comprise first portion 310 for connecting to another instrument, such as a rotary power tool and second portion 312 for connecting to distal reamer 304. Distal reamer 304 can comprise shaft 314 around which middle broach 308 and proximal reamer 306 can be attached and distal reaming head 316. Proximal reamer 306 can comprise hub 318 for receiving shaft 314 and proximal reaming head 320 having blades 321. Middle broach 308 can comprise hub 322 for receiving shaft 314 and blades 324.

Prosthesis implant instrument 300 can be configured to perform multiple bone-modifications in a single operation using the integrated bone-modifying components of distal reamer 304, middle broach 308 and proximal reamer 306. Distal reamer 304, middle broach 308 and proximal reamer 306 can be configured to make different recesses or voids within bone matter to receive different parts of a prosthetic implant, such as arthroplasty system 200 of FIG. 3. In examples, distal reamer 304 can be configured to make a center bore or boss for receiving central hub 130 (FIG. 6), middle broach 308 can be configured to make spoke slots extending from the center bore to receive fin 101 through fin 106 (FIG. 4) and proximal reamer 306 can be configured to make a socket for receiving tray 204 (FIG. 3).

In examples, the distal tips of blades 324 of middle broach 308 can be engaged with a bone surface. Blades 324 of middle broach 308 can be inserted into bone matter and prosthesis implant instrument 300 can be pushed into the bone surface so that distal reaming head 316 of distal reamer 304 contacts the bone surface. Coupler 302 can be rotated by drive tool 402 to cause rotation of shaft 314 and distal reaming head 316. Shaft 314 can rotate within middle broach 308 and distal reaming head 316 can rotate to remove bone. Engagement of blades 324 with bone matter can prevent rotation of middle broach 308. Prosthesis implant instrument 300 can continue to be advanced into the bone surface, such as by impacting strike plate 400 with a hammer or mallet, to bring proximal reamer 306 into engagement with the bone surface.

Proximal reaming head 320 of proximal reamer 306 can be rotated and advanced into the bone surface to remove bone matter. Distal reamer 304 and proximal reamer 306 can be configured to rotate together while middle broach 308 can be held stationary independent of rotation of distal reamer 304 and proximal reamer 306.

FIG. 10 is a perspective view of distal reamer 304 of FIG. 7 comprising shaft 314 and distal reaming head 316. FIG. 11 is a cross-sectional view of distal reamer 304 of FIG. 10. FIG. 10 and FIG. 11 are discussed concurrently.

Shaft 314 can comprise threaded end 330, cylindrical portion 332, threaded portion 334, boss 336 and shoulder 338. Distal reaming head 316 can comprise body 340, cutting teeth 342, lands 344 and proximal surface 346. Distal reamer 304 can include internal lumen 348 extending therethrough from threaded end 330 to cutting teeth 342.

Distal reamer 304 can comprise a single-piece or monolithic body fabricated from metallic material, such as stainless steel. Distal reamer 304 can be rigid to allow for rotational input to be transmitted therethrough. In examples, threaded end 330 can include right-hand threading for mating with coupler 302 and threaded portion 334 can include left-hand threading for mating with proximal reamer 306. As such, right hand drive input at coupler 302 will have a tendency to tighten the connection at threaded end 330 and coupler 302, and engagement of proximal reamer 306 while rotating in the right-hand direction with bone will not be loosened by rotation of cylindrical portion 332 therein. Cylindrical portion 332 can be long enough to allow proximal reamer 306, middle broach 308 and distal reamer 304 to be inserted through soft tissue, e.g., skin and muscle, to reach bone matter. Boss 336 can have a larger diameter than cylindrical portion 332 to allow boss 336 to extend outward of threaded portion 334 to provide a surface for flush or tight engagement with body 370 of proximal reamer 306 and hub 322 of middle broach 308. Distal reamer 304 can include internal lumen 348 to allow another instrument, such as a pin, to be inserted therein. Distal reaming head 316 can extend radially outward of boss 336 to allow distal reaming head 316 to cut a bone bore the desired size or diameter.

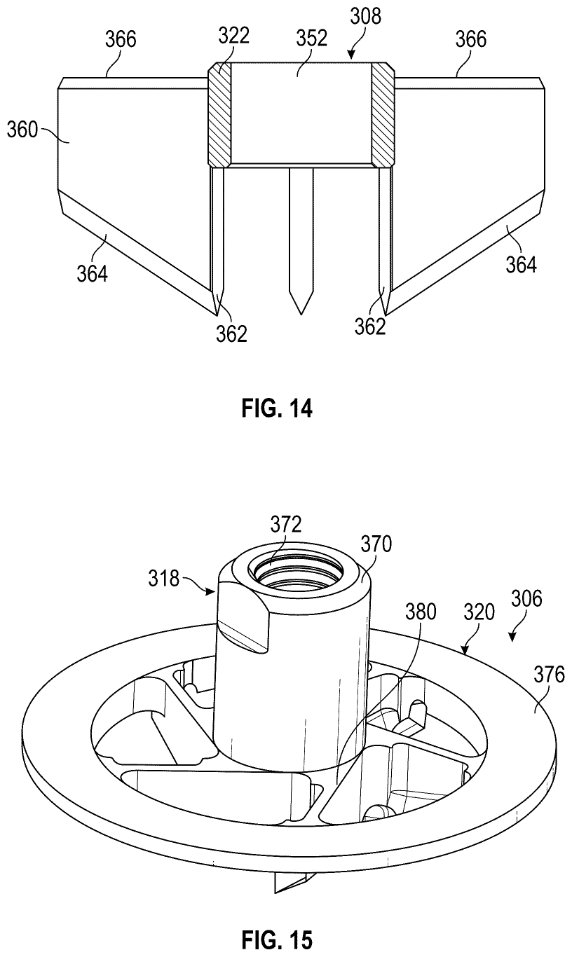

FIG. 12 is a perspective view of middle broach 308 of FIG. 7 comprising hub 322 and blades 324. FIG. 13 is a top view of middle broach 308 of FIG. 12. FIG. 14 is a cross-sectional view of middle broach 308 of FIG. 12. FIG. 12 through FIG. 14 are discussed concurrently.

Hub 322 can comprise an annular body having outer surface 350, bore 352, proximal end 354 and distal end 356. Blades 324 can comprise plate-like bodies 360 having inner ends 362, distal ends 364 and upper ends 366.

Middle broach 308 can comprise a single-piece or monolithic body fabricated from metallic material, such as stainless steel. Middle broach 308 can be rigid to allow for axial pushing though bone matter. Hub 322 can comprise an elongate sleeve or a hub to provide stability and concentricity with boss 336. Bore 352 can be sized to fit tightly around boss 336, but to allow relative rotation therebetween. The thickness of hub 322 between bore 352 and outer surface 350 can be approximately equal to the distance that distal reaming head 316 extends radially outward of boss 336 so that hub 322 does not interfere with the bone bore formed by distal reaming head 316. The shape of blades 324 can be configured to match the shape of fin 101 through fin 106 of humeral anchor 100. For example, the thickness of blades 324 between opposing surfaces of plate-like bodies 360 can be the same thickness as fin 101 through fin 106. In examples, plate-like bodies 360 can have trapezoidal shapes. In examples, the thickness of blades 324 can be slightly less to allow for fin 101 through fin 106 to fit tightly into the radial bone slots formed by blades 324. The angle of distal ends 364 can be the same as the angle on bottom edges 160 of fin 101 through fin 106. Inner ends 362 of blades 324 can be flush with, e.g., axially aligned with, outer surface 350 of hub 322 to allow the radial bone slots formed by blades to be contiguous with the center bone bore or boss formed by distal reaming head such that a single opening in the bone is produced. Upper ends 366 of blades 324 can be below proximal end 354 of hub 322 to reduce the potential for interference with proximal reamer 306. In the illustrated example, blades 324 have the same shape. However, each of blades 324 can have a different shape or can be grouped into similar shapes to match the shapes of fin 101 through fin 106.

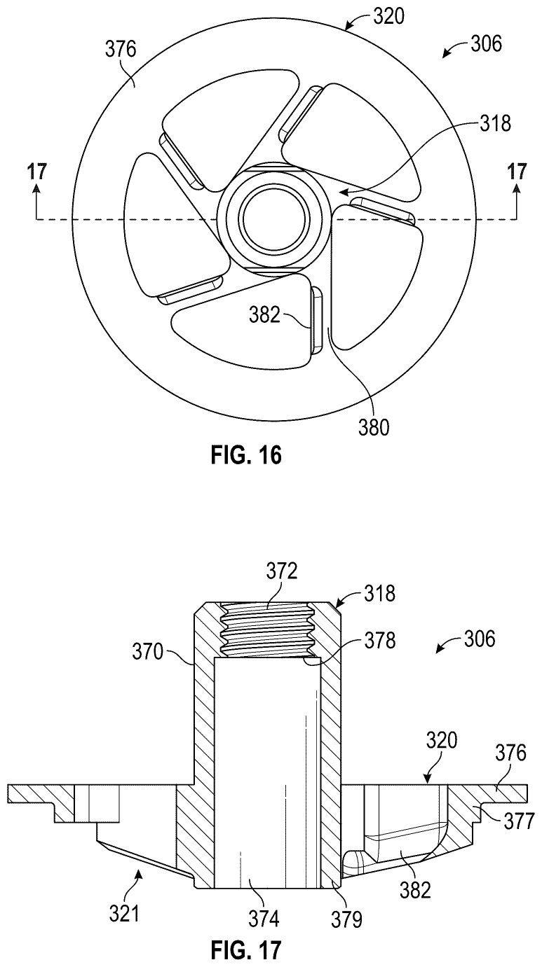

FIG. 15 perspective view of proximal reamer 306 of FIG. 7 comprising hub 318, proximal reaming head 320 and blades 321. FIG. 16 is a top view of proximal reamer 306 of FIG. 15. FIG. 17 is a cross-sectional view of proximal reamer 306 of FIG. 15. FIG. 15 through FIG. 17 are discussed concurrently.

Hub 318 can comprise body 370, bore 372, socket 374, rim 376, shoulder 378 and distal surface 379. Blades 321 can comprise spokes 380 and cutting teeth 382.

Proximal reamer 306 can comprise a single-piece or monolithic body fabricated from metallic material, such as stainless steel. Proximal reamer 306 can be rigid to allow for rotational input to be transmitted therethrough. Body 370 can comprise an elongate sleeve or a hub to provide stability and concentricity with boss 336. As mentioned, socket 374 can be sized to fit around boss 336 and bore 372 can be configured to have left-hand threading or threading opposite that of threaded end 330, e.g., reverse threading. Blades 321 can extend radially outward from body 370 and can be angled to facilitate rotation against bone in a right-hand direction, e.g., clockwise direction looking at FIG. 16. For example, blades 321 can be angled forward in the clockwise direction from body 370 to position cutting teeth 382 for cutting through bone. Teeth 382 can comprise sharpened edges along blades 321 configured to cut through bone matter. In examples, the distal end of blades 321 are angled relative to the rotational axis of proximal reamer 306. However, the distal end of blades 321 can be horizontal. In the illustrated example, five of blades 321 are included. However, fewer or greater numbers can be used. Blades 321 can be configured to form a bone socket into which a tray will fit. For example, blades 321 can form a bone socket to receive bottom surface 208 of tray 204 (FIG. 3). Rim 376 can extend from blades 321. Rim 376 can be radially longer than blades 321 to form ledge 377. Rim 376 can be configured to provide a stop for prosthesis implant instrument 300. For example, rim 376 can be configured to engage resected surface 210 (FIG. 3) radially outside of where prosthesis implant instrument 300 has made bone modifications. Thus, engagement of rim 376 with resected surface 210 can prohibit further axial movement of prosthesis implant instrument 300, thereby setting the depth for distal reamer 304, proximal reamer 306 and middle broach 308. As discussed herein, distal surface 379 can form an upper boundary for middle broach 308.

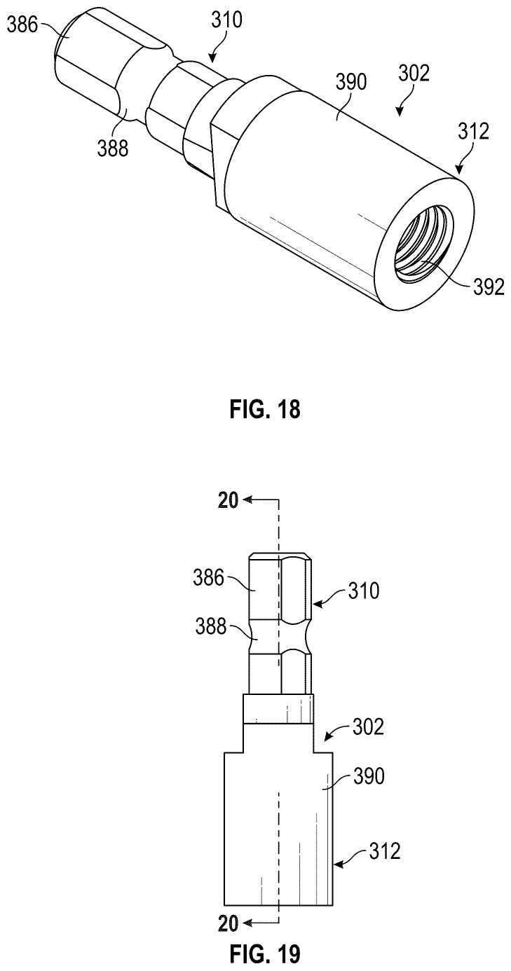

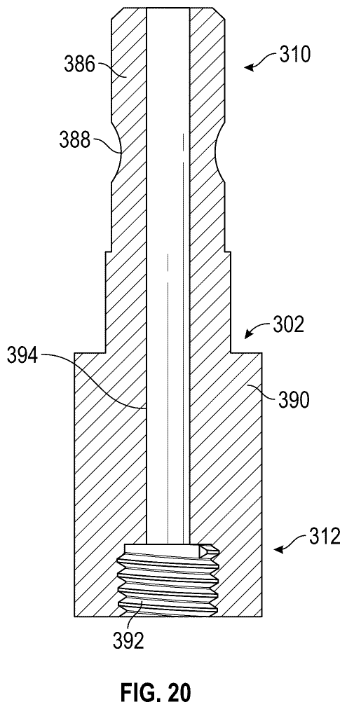

FIG. 18 perspective view of coupler 302 of FIG. 7 showing first portion 310 and second portion 312. FIG. 19 is a side view of the coupler of FIG. 18. FIG. 20 is a cross-sectional view of the coupler reamer of FIG. 18. FIG. 18 through FIG. 20 are discussed concurrently.

First portion 310 can comprise rotational coupling 386 and groove 388. Second portion 312 can comprise barrel 390, socket 392 and bore 394.

First portion 310 can be configured to receive a rotary input and output a similar rotary output. In examples, first portion 310 can comprise a hex head or another body having flat surfaces to receive torque. In the illustrated example, first portion 310 comprises a cylindrical body with three planar surfaces formed therein. Groove 388 can be configured to allow a rotary input device to lock onto coupler 302 or to otherwise prevent or inhibit axial displacement of the rotary input device from coupler 302 during use. Second portion 312 can have an outer diameter larger than the outer diameter of first portion 310. Thus, second portion 312 can form a ledge to engage a strike plate, for example. Socket 392 can be configured to attach to distal reamer 304. Specifically, threaded end 330 of shaft 314 on distal reamer 304 can be inserted into socket 392. Threaded end 330 and socket 392 can be configured to have mating threading. Threaded engagement of threaded end 330 and socket 392 can facilitate torque transmission therebetween. Bore 394 can extend through coupler 302 and can be configured to align with internal lumen 348 when assembled with distal reamer 304.

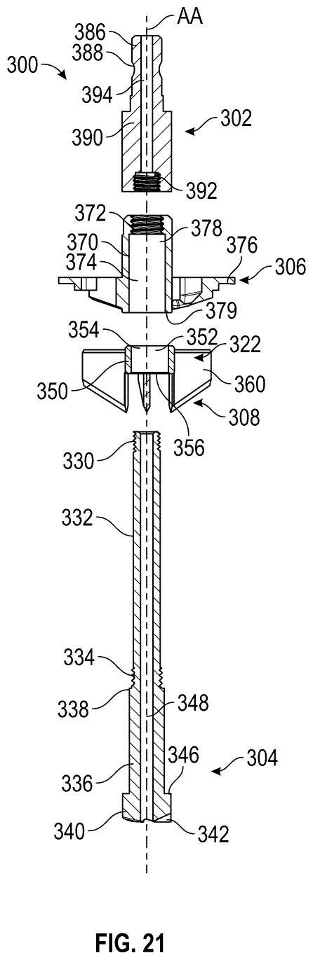

FIG. 21 is an exploded cross-sectional view of prosthesis implant instrument 300 of FIG. 9. Prosthesis implant instrument 300 can extend along axis AA.

To assemble prosthesis implant instrument 300, middle broach 308 can be positioned around distal reamer 304. Specifically, cylindrical portion 332 of shaft 314 of distal reamer 304 can be inserted into bore 352 of middle broach 308. Hub 322 can slide toward distal reaming head 316 around boss 336. Hub 322 can be positioned against proximal surface 346 of distal reaming head 316. Bore 352 and boss 336 can be sized to allow middle broach 308 to rotate or spin about axis AA on distal reamer 304. In examples, bore 352 and boss 336 can form a tight running fit.

With middle broach 308 positioned on distal reamer 304, proximal reamer 306 can be assembled to distal reamer 304. Specifically, cylindrical portion 332 of shaft 314 of distal reamer 304 can be inserted into socket 374 in body 370. Body 370 can be slid toward middle broach 308 and distal reaming head 316 around boss 336. Body 370 can be slid down on distal reamer 304 until shoulder 378 faces shoulder 338. Threading on bore 372 can be engaged with threading on threaded portion 334. Threading on bore 372 and threaded portion 334 can have counter threading or left-handed threading to counteract rotational input provided at coupler 302 in a right-handed direction, thereby preventing proximal reamer 306 from uncoupling or unscrewing from distal reamer during operation. When bore 372 and threaded portion 334 are fully engaged, distal surface 379 of body 370 can be positioned spaced apart from proximal surface 346 a distance that is greater than the height of hub 322. As such, hub 322 can be positioned between proximal surface 346 and distal surface 379 and can be allowed a small amount of axial travel during use.

In examples, assembly of proximal reamer 306 with distal reamer 304 can be omitted, such as to perform an onlay reaming and broaching process. In such examples, a threaded nut having mating threading with threaded portion 334 can be attached to distal reamer to lock middle broach 308 in place. The threaded nut can have a diameter approximately equal to or less than distal reaming head 316 to not interfere with bone matter.

The illustrated example shows middle broach being positioned axially between distal reaming head 316 and proximal reaming head 320. However, prosthesis implant instrument 300 can be arranged in other configurations. For example, prosthesis implant instrument 300 can be configured to have a distal reaming element, a middle reaming element and a proximal broach. In such an example, proximal reamer 306 can lock onto distal reamer 304 as illustrated, and hub 322 of middle broach 308 can be configured to fit around body 370 and axially locked in place via a nut that can attach to a proximal end of body 370. In examples, prosthesis implant instrument 300 can be configured to have a distal broach, a middle reaming element and a proximal reaming element. In such examples, a distal broach can be configured to be inserted into internal lumen 348 and distal reamer 304 and proximal reamer 306 assembled as described can rotate about the reamer.

Coupler 302 can be attached to the proximal end of cylindrical portion 332 of shaft 314 of distal reamer 304. In particular, threaded end 330 of cylindrical portion 332 can be inserted into socket 392. Socket 392 can include threading to engage with threading of threaded end 330. In examples, threading on socket 392 and threading on threaded end 330 can have right-hand threading.

A drive device, such as drive tool 402 (FIG. 7), can be attached to coupler 302 to cause rotation of shaft 314 of distal reamer 304. For example, a socket of a rotary drive tool, such as motorized power tool, can be coupled to rotational coupling 386. The socket and rotational coupling 386 can have mating surfaces that can transmit rotational output of the socket to rotational coupling 386. A detent, such as a ball bearing, of the socket can engage groove 388 to inhibit axial displacement between the socket and rotational coupling 386 during rotation.

Additionally, a strike plate, such as strike plate 400 (FIG. 7), can be connected to coupler 302 proximal of the drive input. The strike plate can comprise a plate or another device to allow an axial force to be transmitted to shaft 314 along or generally along axis AA. As such, the strike plate can comprise a circular disk that enlarges the radial profile of coupler 302 or shaft 314. In examples, the strike plate can be allowed to rotate about axis AA independent of coupler 302. For example, the strike plate can include a central bushing or bearings that allow the strike plate to rotate relative to a cylindrical portion of coupler 302 or shaft 314. Thus, an axial load can be delivered to the strike plate while shaft 314 is rotating.

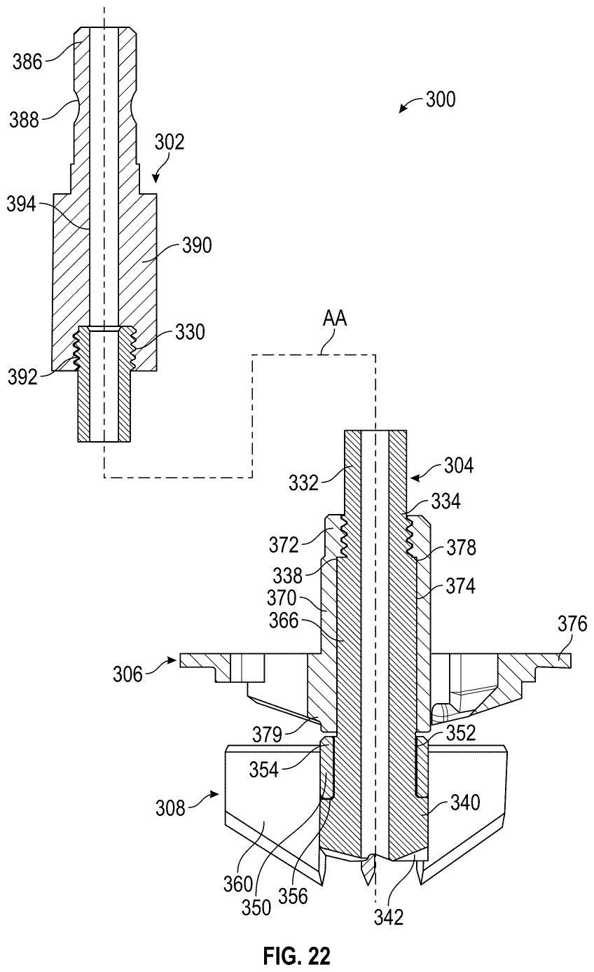

FIG. 22 is a cross-sectional view of prosthesis implant instrument 300 of the present disclosure in an assembled state. Prosthesis implant instrument 300 can extend along axis AA. Prosthesis implant instrument 300 can be configured to perform multiple bone-modifications along axis AA in a single operation using the integrated bone-modifying components of distal reamer 304, middle broach 308 and proximal reamer 306. Distal reamer 304, middle broach 308 and proximal reamer 306 can be configured to make different recesses or voids within bone matter to receive different parts of a prosthetic implant, such as arthroplasty system 200 of FIG. 3. In examples, distal reaming head 316 can be advanced into bone along axis AA to form a cylindrical bone bore into which central hub 130 (FIG. 6) can be positioned. Distal reaming head 316 can be configured to make a bone boss immediately surrounding axis AA of a first diameter. In examples, blades 324 can be advanced into bone matter surrounding the bone bore formed by distal reaming head 316 to receive fin 101 through fin 106 (FIG. 4). Blades 324 of middle broach 308 can extend radially outward of distal reaming head 316 and can partially overlap with distal reaming head 316 in the axial direction. However, blades 324 can be axially paced from distal reaming head 316, e.g., fully above distal reaming head 316. In examples, blades 321 of proximal reamer 306 can be configured to make a concave socket for receiving tray 204 (FIG. 3). The concave socket can comprise a counterbore surrounding the distal, center boss made by distal reaming head 316. Blades 321 can be configured to extend radially outward of distal reaming head 316 and can be positioned axially above and spaced apart from distal reaming head 316. In examples, blades 321 can be radially longer than blades 324, but can be the same radial length or shorter in other examples. Similarly, blades 321 and blades 324 can be configured to axially overlap in other examples.

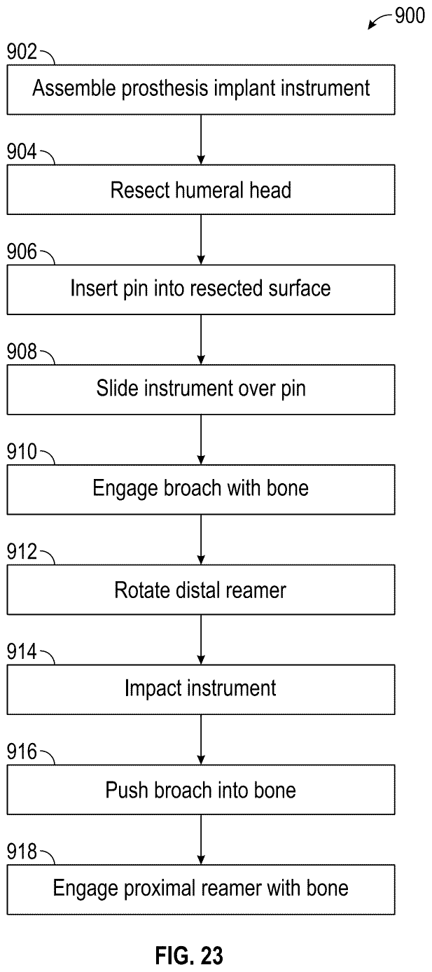

FIG. 23 is a line diagram illustrating method 900 including operations 902 through operation 918 of the present disclosure relating to implanting stemless anchor 202 and tray 204 into resected surface 210, as shown in FIG. 3. Method 900 can comprise operation 902 through operation 918 that describe various operations for operation of prosthesis implant instrument 300. In various examples, additional operations consistent with the devices, systems methods and operations described herein can be included. Likewise, some of operation 902 through operation 918 can be omitted or performed in other sequences.

At operation 902, a prosthesis implant instrument such as the one-step instruments for implanting stemless prostheses of the present disclosure can be assembled. For example, Prosthesis implant instrument 300 can be assembled according to the description provided with reference to FIG. 22. In examples, prosthesis implant instrument 300 can be assembled for two-level bone modification by assembly of distal reamer 304 and middle broach 308. In examples, prosthesis implant instrument 300 can be assembled for three-level bone modification by assembly of distal reamer 304, middle broach 308 and proximal reamer 306. In examples, prosthesis implant instrument 300 can be assembled after operation 904 after the resected humeral bone is evaluated by a surgeon. In examples, prosthesis implant instrument 300 can be pre-assembled before an arthroplasty procedure, such as at a factory or at a medical facility such as a hospital.

At operation 904, a humeral bone can be resected to remove all or a portion of the humeral head to form a resected surface. For example, humerus 10 of FIG. 3 can be resected with a saw to form resected surface 210. Resected surface 210 can form a generally flat, planar surface into which stemless anchor 202 can be positioned.

At operation 906, a pin can be positioned in the resected surface. A guide body, such as a pin, a Steinmann pin, a K wire or a rod, can be inserted in resection surface along a desired axis of rotation. A sizer can be used to place a pin at the center or central portion of resected surface 210. A surgeon can measure the cross-sectional area or diameter of resected surface 210 to determine an appropriate size, e.g., small, medium or large, of prosthetic components to be implanted in humerus 10.

At operation 908, the prosthesis implant instrument assembled at operation 902 can slid over the guide body. For example, a pin can be inserted into internal lumen 348 of distal reamer 304.

At operation 910, the distal tip of prosthesis implant instrument 300 can be engaged with the resected bone surface. For example, the distal tip portion of middle broach 308 can be inserted into resected surface 210. In examples, middle broach 308 can be inserted into bone until distal reamer 304 contacts resected surface 210.

At operation 912, the distal reamer can be rotated. For example, a power tool, such as drive tool 402 (FIG. 7), can be attached to coupler 302 at rotational coupling 386. The power tool can transfer rotational motion to coupler 302, which can transmit the rotational motion to distal reamer 304. Rotation of distal reamer 304 can cause removal of bone from resected surface 210. Engagement of blades 324 with resected surface can prevent middle broach 308 from rotating. For example, shaft 314 of distal reamer 304 can rotate within hub 322.

At operation 914, an impaction instrument can be applied to the prosthesis implant instrument. In examples, an impaction instrument, such as a hammer or mallet, can be impacted directly to a portion of the power tool such that axial force and rotational force can be applied simultaneously or alternatively. In examples, prosthesis implant instrument 300 can be impacted through a strike plate, e.g., strike plate 400 (FIG. 7), connected to prosthesis implant instrument 300 or the power tool. In examples, a strike plate can comprise a body extending from prosthesis implant instrument 300 or attached thereto that can allow for force form an impact instrument to be transmitted along shaft 314 of distal reamer 304. Delivery of impaction to prosthesis implant instrument 300 can cause middle broach 308 to advance further into resected surface 210 and can allow distal reamer 304 to ream deeper into resected surface 210.

At operation 916, the middle broach can be fully inserted into bone below resected surface 210. Impaction of prosthesis implant instrument 300 can allow middle broach 308 to be driven deeper into bone.

At operation 918, the proximal reamer can be engaged with bone. Proximal reamer 306 can be engaged with resected surface 210 radially outward of distal reamer 304. Cutting teeth 382 can be engaged with bone matter in resected surface 210. Rotational input can be imparted to proximal reamer 306 through distal reamer 304. For example, threaded engagement between threaded portion 334 and bore 372 can cause proximal reamer 306 to rotate with distal reamer 304 in a fixed relationship. Prosthesis implant instrument 300 can be axially advanced until rim 376 of proximal reamer 306 engages resected surface 210.

Method 900 is described with reference to implanting a humeral implant, but can be performed in other anatomic locations such as on humeral heads.

The present disclosure provides a one-step instrument designed for implanting stemless prostheses, particularly in the context of shoulder arthroplasty surgeries. The instrument integrates multiple functions, including reaming and broaching, to prepare the humerus bone or another bone to receive a prosthetic implant, such as those having variable radial and axial geometry.

The systems, devices and methods discussed in the present application can be useful and provide advantages in the following ways:
Bone Preservation: The instrument is designed to preserve bone matter by avoiding the need for elongate stems in the humeral canal, which is particularly beneficial in stemless humeral implants.
Surgical Simplicity: By combining reaming and broaching operations into a single device, the instrument simplifies the surgical procedure, making it easier to assemble and operate.
Reduced Complexity: The integration of multiple bone modification tools into one instrument reduces the complexity of the surgery, potentially shortening the duration of the procedure and lowering overall costs.
Versatility: The instrument can be used for both onlay and inlay bone preparation and is applicable to various prosthetic components, including stemmed and stemless options.
Reduced Trauma: The design of the instrument aims to minimize additional bone removal and, consequently, reduce trauma and post-operative pain for the patient.
Enhanced Recovery: By alleviating pain and providing an increased range of motion at the shoulder joint, the instrument contributes to an enhanced recovery process for patients undergoing partial or total shoulder arthroplasty.
Customizable Depth: The instrument allows for the adjustment of the depth of implantation, which is particularly useful in reverse shoulder systems to control joint tightness.
Efficient Design: The instrument's design, which includes a distal reamer, a middle broach, and a proximal reamer, allows for the efficient preparation of the bone in a single procedure.
Improved Outcomes: The one-step instrument aims to provide improved surgical outcomes by integrating multiple functions, reducing the risk of errors, and ensuring a more consistent and reliable process for implanting prostheses.
Overall, the invention presents a significant advancement in the field of medical instruments for shoulder arthroplasty, offering a more streamlined, efficient, and less invasive approach to implanting stemless prostheses.

### EXAMPLES

Example 1 is an instrument for preparing a bone to receive a prosthetic implant, the instrument comprising: a shaft extending along a central axis from a distal end to a proximal end; a distal reaming element located at the distal end of the shaft, wherein the distal reaming element comprises a boss reamer; and a broaching element located proximally of the distal reaming element, the broaching element configured to rotate about the shaft, wherein the broaching element comprises a plurality of spokes extending radially outward of the distal reaming element.

In Example 2, the subject matter of Example 1 optionally includes a proximal reaming element located proximal of the distal reaming element.

In Example 3, the subject matter of Example 2 optionally includes wherein the proximal reaming element comprises: a central hub configured to fit around the shaft; and one or more cutting features extending radially from the central hub.

In Example 4, the subject matter of Example 3 optionally includes a disk element extending surrounding the central hub; wherein the one or more cutting features connect the disk element and the central hub.

In Example 5, the subject matter of Example 4 optionally includes wherein the one or more cutting features of the proximal reaming element extend distally from the disk element and are shaped to produce a concave compartment in a resected bone surface.

In Example 6, the subject matter of any one or more of Examples 4-5 optionally include wherein the one or more cutting features extending distally of the disk element and are located proximally of the broaching element.

In Example 7, the subject matter of any one or more of Examples 2-6 optionally include wherein a distal tip of the broaching element extends distally of the distal reaming element in an axial direction.

In Example 8, the subject matter of Example 7 optionally includes wherein the distal reaming element comprises a distal end face of the shaft.

In Example 9, the subject matter of Example 8 optionally includes wherein the distal reaming element comprises a cylindrical body located radially inward of the broaching element.

In Example 10, the subject matter of any one or more of Examples 2-9 optionally include wherein the broaching element comprises: a center hub configured to fit around the shaft; and a plurality of blade tips disposed on each of the plurality of spokes.

In Example 11, the subject matter of Example 10 optionally includes wherein each of the plurality of spokes comprises a trapezoidal shaped plate.

In Example 12, the subject matter of Example 11 optionally includes wherein the shaft comprises a shoulder for engaging the center hub.

In Example 13, the subject matter of Example 12 optionally includes wherein the proximal reaming element attaches to the shaft in a fixed relationship.

In Example 14, the subject matter of Example 13 optionally includes wherein the proximal reaming element attaches to the shaft via reverse-threaded coupling.

In Example 15, the subject matter of any one or more of Examples 13-14 optionally include wherein the proximal reaming element is spaced from the distal reaming element to form a slot for receiving the broaching element.

In Example 16, the subject matter of any one or more of Examples 2-15 optionally include wherein: the broaching element is configured to broach slots for an anchoring element of a stemless shoulder implant; and the proximal reaming element is configured to ream a counterbore for a tray for a head of the stemless shoulder implant.

In Example 17, the subject matter of any one or more of Examples 1-16 optionally include wherein the shaft comprises a central lumen.

In Example 18, the subject matter of any one or more of Examples 1-17 optionally include wherein the proximal end of the shaft comprises a coupler for a rotary input tool, the coupler configured to transmit rotational force.

Example 19 is a method of modifying a humeral head to receive a stemless humeral implant using an instrument having an integrated broach and reamer, the method comprising: resecting a bone to form a resected surface; inserting the instrument into the resected surface; rotating a shaft of the instrument to rotate a distal reamer and form a center boss; and advancing a broach of the instrument into the resected surface to form a plurality of slots extending from the center boss.

In Example 20, the subject matter of Example 19 optionally includes wherein inserting the instrument into the resected surface comprises partially inserting the broach of the instrument before engaging the distal reamer with the resected surface.

In Example 21, the subject matter of Example 20 optionally includes preventing rotation of the broach via engagement with bone matter below the resected surface.

In Example 22, the subject matter of any one or more of Examples 19-21 optionally include rotating a proximal reamer with the shaft; and advancing the broach and the proximal reamer into the resected surface.

In Example 23, the subject matter of Example 22 optionally includes rotating the shaft of the instrument to simultaneously rotate the proximal reamer and the distal reamer.

In Example 24, the subject matter of any one or more of Examples 22-23 optionally include forming a proximal bone compartment in the resected surface with the proximal reamer at a proximal end of the center boss; and forming the plurality of slots extending from the center boss distally of the proximal bone compartment with the broach.

In Example 25, the subject matter of any one or more of Examples 19-24 optionally include impacting a proximal portion of the instrument to advance the broach into the resected surface.

In Example 26, the subject matter of Example 25 optionally includes simultaneously impacting and rotating the distal reamer.

In Example 27, the subject matter of any one or more of Examples 19-26 optionally include inserting a pin into the resected surface; and sliding the shaft of the instrument over the pin.

Each of these non-limiting examples can stand on its own, or can be combined in various permutations or combinations with one or more of the other examples.

### VARIOUS NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An instrument for preparing a bone to receive a prosthetic implant, the instrument comprising:
a shaft extending along a central axis from a distal end to a proximal end;
a distal reaming element located at the distal end of the shaft, wherein the distal reaming element comprises a boss reamer; and
a broaching element located proximally of the distal reaming element, the broaching element configured to rotate about the shaft, wherein the broaching element comprises a plurality of spokes extending radially outward of the distal reaming element.

2. The instrument of claim 1, further comprising a proximal reaming element located proximal of the distal reaming element.

3. The instrument of claim 2, wherein the proximal reaming element comprises:
a central hub configured to fit around the shaft;
one or more cutting features extending radially from the central hub; and
a disk element extending surrounding the central hub;
wherein the one or more cutting features connect the disk element and the central hub.

4. The instrument of claim 3, wherein the one or more cutting features of the proximal reaming element extend distally from the disk element and are shaped to produce a concave compartment in a resected bone surface.

5. The instrument of claim 3, wherein the one or more cutting features extending distally of the disk element and are located proximally of the broaching element.

6. The instrument of claim 2, wherein a distal tip of the broaching element extends distally of the distal reaming element in an axial direction.

7. The instrument of claim 6, wherein the distal reaming element comprises a cylindrical body forming a distal end face of the shaft that is located radially inward of the broaching element.

8. The instrument of claim 2, wherein the broaching element comprises:
a center hub configured to fit around the shaft; and
a plurality of blade tips disposed on each of the plurality of spokes.

9. The instrument of claim 8, wherein:
each of the plurality of spokes comprises a trapezoidal shaped plate;
the shaft comprises a shoulder for engaging the center hub; and
the proximal reaming element attaches to the shaft in a fixed relationship.

10. The instrument of claim 9, wherein the proximal reaming element attaches to the shaft via reverse-threaded coupling.

11. The instrument of claim 9, wherein the proximal reaming element is spaced from the distal reaming element to form a slot for receiving the broaching element.

12. The instrument of claim 2, wherein:
the broaching element is configured to broach slots for an anchoring element of a stemless shoulder implant; and
the proximal reaming element is configured to ream a counterbore for a tray for a head of the stemless shoulder implant.

13. The instrument of claim 1, wherein the shaft comprises a central lumen.

14. The instrument of claim 1, wherein the proximal end of the shaft comprises a coupler for a rotary input tool, the coupler configured to transmit rotational force.

15. A method of modifying a humeral head to receive a stemless humeral implant using an instrument having an integrated broach and reamer, the method comprising:
resecting a bone to form a resected surface;
inserting the instrument into the resected surface;
rotating a shaft of the instrument to rotate a distal reamer and form a center boss; and
advancing a broach of the instrument into the resected surface to form a plurality of slots extending from the center boss.

16. The method of claim 15, wherein:
inserting the instrument into the resected surface comprises partially inserting the broach of the instrument before engaging the distal reamer with the resected surface; and
rotating the shaft of the instrument to rotate the distal reamer and form the center boss comprises preventing rotation of the broach via engagement with bone matter below the resected surface.

17. The method of claim 15, further comprising:
rotating a proximal reamer with the shaft;
advancing the broach and the proximal reamer into the resected surface; and
rotating the shaft of the instrument to simultaneously rotate the proximal reamer and the distal reamer.

18. The method of claim 17, further comprising:
forming a proximal bone compartment in the resected surface with the proximal reamer at a proximal end of the center boss; and
forming the plurality of slots extending from the center boss distally of the proximal bone compartment with the broach.

19. The method of claim 15, further comprising:
impacting a proximal portion of the instrument to advance the broach into the resected surface; and
simultaneously impacting and rotating the distal reamer.

20. The method of claim 15, further comprising:
inserting a pin into the resected surface; and
sliding the shaft of the instrument over the pin.
